(19) United States
(12) Patent Application Publication
(10) Pub. No.: US 2023/0052487 A1
(43) Pub. Date: Feb. 16, 2023

(54) FOAMING BODY DEODORIZER

(71) Applicant: Alhaji Sesay, Los Angeles, CA (US)

(72) Inventor: Alhaji Sesay, Los Angeles, CA (US)

(21) Appl. No.: 17/884,496

(22) Filed: Aug. 9, 2022

Related U.S. Application Data
(60) Provisional application No. 63/233,605, filed on Aug. 16, 2021.

(51) Int. Cl. A61K 8/9789; A61K 8/04; A61K 8/365; A61K 8/9794; A61K 8/60; A61K 8/22; A61Q 15/00

(52) U.S. Cl. CPC A61K 8/9789; A61K 8/046; A61K 8/365; A61K 8/9794; A61K 8/60; A61K 8/22; A61Q 15/00; A61K 2800/87

(57) ABSTRACT

A method of making a foaming deodorant containing an aqueous solution of citric acid, aloe vera gel, capryl capryl glucose, hydrogen peroxide, and witch hazel. The foaming deodorant may also have an essential oil, a fragrance, and/or a color. The foaming deodorant is added to a dispensing container and dispensed from a pressurized dispensing container or from a dispensing container having a dispensing pump. Citric acid and hydrogen peroxide safely kill bacteria on the skin. The foaming deodorant may be used anywhere on the skin of the body.

### RELATED APPLICATIONS

This application claims the benefit of a US patent application that was filed on Aug. 16, 2021, as 63/233,605, which application is incorporated by reference in its entirety.

### FIELD OF THE DISCLOSURE

The present invention relates to deodorants and, more particularly, to an odor neutralizing foam or mousse pre-wash astringent that deodorizes the body by killing bacteria that causes body odor. The foaming deodorant is an acidic solution that kills malodor causing bacteria, lifting and neutralizing odor.

### BACKGROUND

The benefits of deodorants are well-known. Deodorants are used to minimize unpleasant odors on the body and can also act as an anti-perspirant at times. Most deodorants are sold in a solid or liquid format in various bottles and containers. Lacking from existing deodorants is an option for a foaming pre-wash astringent and deodorizer that can be used on any part of the body to prevent odor-forming bacteria.

### SUMMARY OF THE INVENTION

This disclosure describes methods of making a foaming deodorant. The methods include forming an aqueous solution of citric acid; adding aloe vera gel to the aqueous solution of citric acid; adding capryl capryl glucose to the aqueous solution of citric acid; adding hydrogen peroxide to the aqueous solution of citric acid; adding witch hazel to the aqueous solution of citric acid; and adding purified water to the aqueous solution of citric acid. The method further includes adding the foaming deodorant to a dispensing container and dispensing the foaming deodorant from a pressurized dispensing container or dispensing the foaming deodorant from a dispensing container having a dispensing pump. The method may also include adding an essential oil, a fragrance, and/or a color to the aqueous solution of citric acid.

An advantage of the foaming deodorant is citric acid which safely kills bacteria by staying on the skin.

Another advantage is hydrogen peroxide which also kills bacteria on the skin.

Another advantage is a foaming deodorant that contains natural ingredients and no harsh chemicals.

Another advantage is a foaming deodorant that may be used anywhere on the skin of the body.

Another advantage is the use of hydrogen peroxide to lift and neutralize odor from the skin.

### BRIEF DESCRIPTION OF THE DRAWINGS

Embodiments of the present disclosure are described in detail below with reference to the following drawings. These and other features, aspects, and advantages of the present disclosure will become better understood with regard to the following description, appended claims, and accompanying drawings. The drawings described herein are for illustrative purposes only of selected embodiments and not all possible implementations and are not intended to limit the scope of the present disclosure.

FIG. 1 depicts a pictorial illustration of a foaming deodorizer according to a non-limiting embodiment.

FIG. 2 depicts a flowchart of an exemplary method for forming the deodorizer.

### DETAILED DESCRIPTION

In the Summary above and in this Detailed Description, and the claims below, and in the accompanying drawings, reference is made to particular features (including method steps) of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

The term “comprises”, and grammatical equivalents thereof are used herein to mean that other components, ingredients, steps, among others, are optionally present. For example, an article “comprising” (or “which comprises”) components A, B, and C can consist of (i.e., contain only) components A, B, and C, or can contain not only components A, B, and C but also contain one or more other components.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

When, in this specification, a range is given as “(a first number) to (a second number)” or “(a first number)-(a second number),” this means a range whose lower limit is the first number and whose upper limit is the second number. For example, 25 to 100 mm means a range whose lower limit is 25 mm and upper limit is 100 mm.

Certain terminology and derivations thereof may be used in the following description for convenience in reference only and will not be limiting. References in the singular tense include the plural, and vice versa, unless otherwise noted.

The present description is drawn to a novel deodorizer that can be produced in the form of a foaming deodorizer. The foaming deodorizer can help to eliminate human odor by killing bacteria that appears on the surface of the skin on any body part or region, including, but not limited to, the under arms (armpits), genitalia (except the vagina), feet, or any other area known for producing and emitting odors on the body. In a non-limiting embodiment, the ingredients of the foaming deodorant include purified water, witch hazel, hydrogen peroxide, citric acid, aloe vera gel, capryl capryl glucoside, essential oil (e.g., peppermint oil), fragrance, and color. Further details are provided with reference to the Figures.

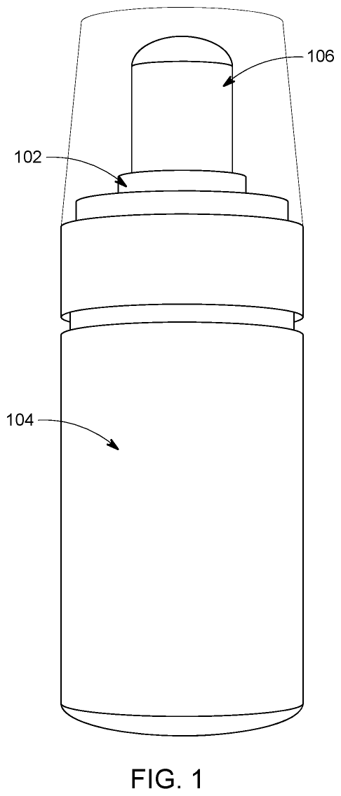

FIG. 1 shows a pictorial illustration of a foaming deodorizer or deodorant 102. In a non-limiting embodiment, the foaming deodorizer 102 is contained as a solution in a foam dispensing container 104 or bottle. The dispensing container may be pressurized (as indicated in FIG. 1) without toxic propellants that automates the dispensing of the foaming deodorizer solution contained within the container 104. In a non-limiting embodiment, the container 104 may include a pump 106 that can pump out the foaming deodorizer solution contained inside the container 104. The container 104 and pump 106, in a non-limiting embodiment, helps to aerate the mixed solution in order to produce the deodorant mixture/solution as a foam that can be applied to the surface of one's skin. In other embodiments, the foaming deodorizer 102 may be contained in an aerosol can that dispenses the foaming deodorant 102.

The foaming deodorant 102 can be first applied to any part of the body that the user wants to eliminate odor. Then, the foaming deodorant 102 may be left on the body part for a minimum period of time, and finally washed off after a minimum period of time has passed. The first time the user applies the foaming deodorant 102, the user may want to allow the foaming deodorant 102 to remain on the skin for 5-10 minutes. After continuous use, there may be less bacteria that needs to be killed by the foaming deodorant 102 and less time is required for the foaming deodorant 102 to be effective (e.g., may be applied for a few minutes and then washed off).

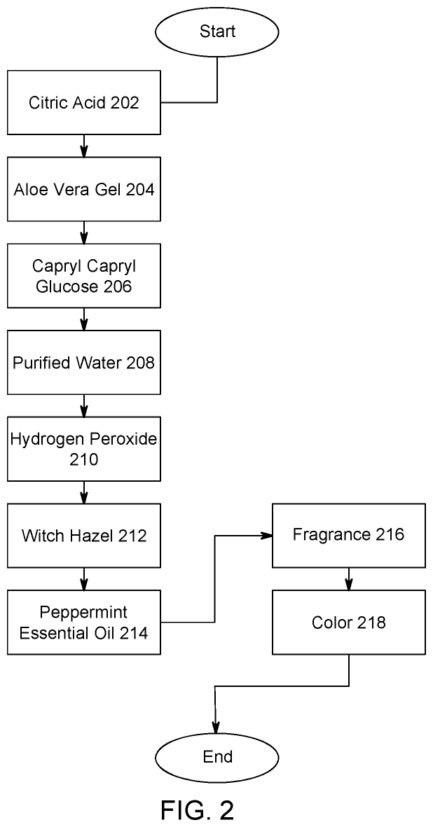

FIG. 2 shows a method of making the foaming deodorant 102 in one or more non-limiting embodiments. In a non-limiting embodiment, the first step 202 may include forming an aqueous solution of citric acid in a mixing container in order to begin the process. Aloe vera gel may be added at step 204. Capryl capryl glucose may be added to the mixture at step 206. Purified water may also be added to the existing mixture at step 208. Hydrogen peroxide may be added at step 210 and witch hazel may then be added to the mixture at step 212. An amount of peppermint oil may be added at step 214 as well as additional fragrance at step 216 and color at step 218. It is noted that in a non-limiting embodiment, the color may be omitted from the solution for the foaming deodorant 102 solution. The solution may be mixed gently together until all of the citric acid dissolves and all of the ingredients or components are mixed into a consistent solution. In this manner, in a non-limiting embodiment, the foaming deodorant 102 solution is formed and may be contained in a foam dispensing container 104 such as that shown in FIG. 1 in an exemplary embodiment.

The citric acid included in the foaming deodorant 102 may aid in safely killing any bacteria on one's skin and may adjust the pH level of the skin to make the area less prone to growth of bacteria. Further, the citric acid may help to exfoliate dead skin cells, dirt, oil, and any deodorant residue. The hydrogen peroxide may further aid in killing bacteria on the skin, neutralizing any odors, as well as aids in penetrating the skin's epidermis with the citric acid included in the mixture (e.g., as shown at step 202).

The capryl capryl glucoside may act as a natural emulsifier that will aid with all of the components in the solution mixing to produce a foam when ejected from the foam dispenser container 104 (e.g., bottle with a pump 106 as shown in FIG. 1). Further, the aloe vera gel that may be added at step 204, as shown in FIG. 2, may further offer healing properties as well as help to thicken the deodorizing solution so that when the foam portion of the solution melts, the remaining amount of the deodorant stays wet and sticks to the skin.

In a non-limiting embodiment, the components of the foaming deodorant 102 may be weighed for appropriate ratios by grams on a scale. In a non-limiting embodiment, the solution may contain the following percentage strength (w/v) of each component: purified water 30% to 40%, preferably 35%; witch hazel 15% to 25%, preferably 20%; hydrogen peroxide 15% to 25%, preferably 20%; citric acid 10% to 20%, preferably 15%; aloe vera gel 1% to 10%, preferably 5%; capryl capryl glucose 1% to 10%, preferably 5%, and a nominal amount (1% or less) of essential oil (e.g., peppermint oil), fragrance, and/or color.

Accordingly, the foaming deodorant 102 is unique because foaming deodorant 102 uses natural ingredients, as described above that can purify lingering body odors caused by trapped bacteria on the skin, leaving the user odorless without harsh chemicals or sticky coverups. The foaming deodorant 102 may be used for the under arms, genitalia, toes, feet, and anywhere on the body and/or skin where odor may be a problem. The user may dispense 1-2 pumps of the foaming deodorant 102 on any area of the body and/or skin that the user wants to deodorize. The foaming deodorant 102 may be massaged into the skin to increase effectiveness and then after a minimum period of time (e.g., 3-5 minutes), may be washed off with soap and water. The foaming deodorant 102 actively works on the top layer of the user's skin to kill any odor-causing bacteria, leaving the user feeling clean and odorless. In a non-limiting embodiment, the foaming deodorant 102 is not an antiperspirant, however, it may provide a fragrance from the user.

The corresponding structures, materials, and acts in the claims below are intended to include any structure, material, or act for performing the function in combination with other claimed elements as specifically claimed. The description of the present invention has been presented for purposes of illustration and description but is not intended to be exhaustive or limited to the invention in the form disclosed. Many modifications and variations will be apparent to those of ordinary skill in the art without departing from the scope and spirit of the invention.

The embodiments were chosen and described in order to best explain the principles of the invention and the practical application, and to enable others of ordinary skill in the art to understand the invention for various embodiments with various modifications as are suited to the particular use contemplated. The present invention according to one or more embodiments described in the present description may be practiced with modification and alteration within the spirit and scope of the appended claims. Thus, the description is to be regarded as illustrative instead of restrictive of the present invention.

What is claimed is:

1. A method of making a foaming deodorant, the method comprising the steps of:
a) preparing a solution of a foaming deodorant; and
b) adding the solution of the foaming deodorant into a dispensing container constructed to dispense the foaming deodorant as a foam.

2. The method of claim 1, wherein the step of preparing the solution of the foaming deodorant comprises:
a) forming an aqueous solution of citric acid;
b) adding aloe vera gel to the aqueous solution of citric acid;
c) adding capryl capryl glucose to the aqueous solution of citric acid;
d) adding hydrogen peroxide to the aqueous solution of citric acid;
e) adding witch hazel to the aqueous solution of citric acid; and
f) adding purified water to the aqueous solution of citric acid.

3. The method of claim 1, further comprising dispensing the foaming deodorant from a pressurized dispensing container.

4. The method of claim 1, further comprising dispensing the foaming deodorant from a dispensing container having a dispensing pump.

5. The method of claim 2, further comprising adding aloe vera gel to the aqueous solution of citric acid in the amount of 1% to 10% w/v, adding capryl capryl glucose to the aqueous solution of citric acid in the amount of 1% to 10% w/v, adding hydrogen peroxide to the aqueous solution of citric acid in the amount of 15% to 25% w/v, adding witch hazel to the aqueous solution of citric acid in the amount of 15% to 25% w/v, and adding water to the aqueous solution of citric acid in the amount 30% to 40% w/v.

6. The method of claim 2, further comprising adding an essential oil, a fragrance, and/or a color in the amount of 1% or less w/v to the aqueous solution of citric acid.

7. A method of making a foaming deodorant, the method comprising the steps of:
a) forming an aqueous solution of citric acid;
b) adding aloe vera gel to the aqueous solution of citric acid;
c) adding capryl capryl glucose to the aqueous solution of citric acid;
d) adding hydrogen peroxide to the aqueous solution of citric acid;
e) adding witch hazel to the aqueous solution of citric acid; and
f) adding purified water to the aqueous solution of citric acid.

8. The method of claim 7, further comprising adding aloe vera gel to the aqueous solution of citric acid in the amount of 1% to 10% w/v, adding capryl capryl glucose to the aqueous solution of citric acid in the amount of 1% to 10% w/v, adding hydrogen peroxide to the aqueous solution of citric acid in the amount of 15% to 25% w/v, adding witch hazel to the aqueous solution of citric acid in the amount of 15% to 25% w/v, and adding water to the aqueous solution of citric acid in the amount 30% to 40% w/v.

9. The method of claim 8, further comprising adding the foaming deodorant to a dispensing container and dispensing the foaming deodorant from a pressurized dispensing container or dispensing the foaming deodorant from a dispensing container having a dispensing pump.

10. The method of claim 8, further comprising adding an essential oil, a fragrance, and/or a color in the amount of 1% or less w/v to the aqueous solution of citric acid.

11. A foaming deodorant produced by a process, comprising:
a) forming an aqueous solution of citric acid;
b) adding aloe vera gel to the aqueous solution of citric acid;
c) adding capryl capryl glucose to the aqueous solution of citric acid;
d) adding hydrogen peroxide to the aqueous solution of citric acid;
e) adding witch hazel to the aqueous solution of citric acid; and
f) adding purified water to the aqueous solution of citric acid.

12. The foaming deodorant of claim 11, further comprising adding aloe vera gel to the aqueous solution of citric acid in the amount of 1% to 10% w/v, adding capryl capryl glucose to the aqueous solution of citric acid in the amount of 1% to 10% w/v, adding hydrogen peroxide to the aqueous solution of citric acid in the amount of 15% to 25% w/v, adding witch hazel to the aqueous solution of citric acid in the amount of 15% to 25% w/v, and adding water to the aqueous solution of citric acid in the amount 30% to 40% w/v.

13. The foaming deodorant of claim 12, further comprising adding the foaming deodorant to a dispensing container and dispensing the foaming deodorant from a pressurized dispensing container or dispensing the foaming deodorant from a dispensing container having a dispensing pump.

14. The method of claim 8, further comprising adding an essential oil, a fragrance, and/or a color in the amount of 1% or less w/v to the aqueous solution of citric acid.
